(19) United States
(12) Patent Application Publication
(10) Pub. No.: US 2024/0360423 A1
(43) Pub. Date: Oct. 31, 2024

(54) METHODS OF IMPROVING RAAV PRODUCTION

(71) Applicant: Genzyme Corporation, Cambridge, MA (US)

(72) Inventors: Srinivas Nellimarla, Lexington, MA (US); Kevin Victor Chen, Sudbury, MA (US); Jeong Lee, Cambridge, MA (US); Carlos Benitez, Cambridge, MA (US); Ying Liu, Cambridge, MA (US)

(73) Assignee: Genzyme Corporation, Cambridge, MA (US)

(21) Appl. No.: 18/648,199

(22) Filed: Apr. 26, 2024

Related U.S. Application Data
(60) Provisional application No. 63/462,209, filed on Apr. 26, 2023.
(60) Provisional application No. 63/462,212, filed on Apr. 26, 2023.

(51) Int. Cl. C12N 7/00; C12N 15/86

(52) U.S. Cl. CPC C12N 7/00; C12N 15/86; C12N 2750/14143; C12N 2750/14151

(57) ABSTRACT

This disclosure relates to methods and compositions for scaling up production of rAAV by cell cultures. In some aspects, the present disclosure relates to methods of transferring a plurality of transfection complexes to a bioreactor. Also provided are methods of improving production of recombinant adeno-associated virus (rAAV) particles by adding a second medium to the cell culture after transfection, thereby improving rAAV particle yield and reducing the amount of time needed to produce high levels of rAAV particles.

### RELATED APPLICATIONS

This application claims the benefit under 35 § 119 (e) of U.S. Provisional Application No. 63/462,209, filed on Apr. 26, 2023, entitled “METHODS OF IMPROVING RAAV PRODUCTION,” and U.S. Provisional Application No. 63/462,212, filed on Apr. 26, 2023, entitled “METHODS OF SCALING UP PRODUCTION OF RAAV” the contents of each of which are hereby incorporated by reference in their entirety.

### BACKGROUND

One effective way to produce viral vectors for therapeutic applications, including but not limited to gene therapy, is to use cultured cells for producing recombinant viral particles. For clinical applications, large quantities of viral particles are required, creating a need to improve quantity and quality of viral (e.g., rAAV) production.

Therefore, there is a need to improve current manufacturing processes for viral vectors use in, for example, gene therapy.

### SUMMARY

This application provides methods and compositions for scaling up and/or improving production of rAAV particles by cultured cells (e.g., using a cell culture). In some aspects, methods and compositions disclosed herein are useful for large scale manufacturing of rAAV and can improve the quantity of rAAV particles produced by cultured cells. In some embodiments, methods and compositions disclosed herein are useful for reducing the risk of contamination during rAAV production (e.g., by reducing the number of batches of components to be combined). In some embodiments, methods and compositions disclosed herein are useful for reducing one or more resources needed to produce rAAV particles. In some embodiments, methods and compositions disclosed herein are useful for decreasing the time required for cells to produce rAAV particles. In some embodiments, methods and compositions disclosed herein are useful to increase the quality of rAAV produced in cell culture (e.g., by increasing the number of full viral particles, assessed, for instance by measuring total viral particles and viral genomes within the total viral particles, to derive the percent full viral particles).

In some aspects, the present disclosure provides a method comprising (a) contacting a cell culture in a first medium with one or more recombinant nucleic acids and a transfection reagent; (b) incubating the cell culture for 1-20 hours (e.g., 2-15 hours, for example 3-10 hours or 4-8 hours) under conditions sufficient for nucleic acid transfection; and (c) contacting the cell culture of (b) with a second medium for improving rAAV production. In some embodiments, the second medium is added about 4-8 hours after step (a). In some embodiments, the second medium is added in two parts, initially about 4-8 hours after step (a) and next about 24 hours after step (a). In some embodiments, the cells are harvested about 48 hours after step (a). In some embodiments, a method of the present disclosure produces a higher titer of rAAV particles. In some embodiments, a method of the present disclosure produces a higher percentage of full capsids. In some embodiments, a method of the present disclosure allows for a shortened production time for rAAV manufacturing.

In some embodiments, incubating the cell culture under conditions sufficient for nucleic acid transfection comprises contacting the cell culture with one or more transfection reagents.

In some embodiments, the method further comprises measuring a level of a metabolite during act (b) above and providing the second medium when the level of the metabolite meets or exceeds a threshold level. In some embodiments, the method further comprises measuring a level of a nutrient during act (b) above and providing the second medium when the level of the nutrient falls at or below a threshold level.

In some embodiments, the cell culture comprises epithelial cells. In some embodiments, the cell culture comprises mammalian cell. In some embodiments, the cell culture comprises HEK cells, CHO cells, or Hela cells. In some embodiments, the cell culture comprises HEK293 cells.

In some embodiments, the method further comprises adding a third medium after the start of act (b) (for example 12-36 hours after, e.g., 24 hours after the start of act (b)).

In some embodiments, the first and second medium are the same medium. In some embodiments, the first medium is different from the second medium.

In some embodiments, the one or more recombinant nucleic acids comprise deoxyribonucleic acid (DNA). In some embodiments, the one or more recombinant nucleic acids comprise ribonucleic acid (RNA). In some embodiments, the one or more recombinant nucleic acids comprise one or more plasmids.

In some embodiments, the cell culture produces adeno-associated virus (AAV) particles. In some embodiments, the method improves production of AAV particles relative to a production method without act (c) above. In some embodiments, the method improves production of AAV particles by at least 2-fold relative to a production method without act (c). In some embodiments, the method improves production of AAV particles by at least 2-fold (for example at least 3-fold, or at least 4-fold, e.g., 4-5 fold, 4-6 fold, 4-7 fold, 4-8 fold, 4-9 fold, or 4-10 fold) relative to a production method without act (c). In some embodiments, the method increases the total number of AAV particles produced relative to a production method without act (c).

In some embodiments, the metabolite is lactate or lactic acid. In some embodiments, the nutrient is a carbon source (e.g., glucose or galactose), amino acid source, trace metal, vitamin, antioxidant source, or mineral.

In some embodiments, the cell culture comprises mammalian cells (such as HEK293 cells) that are transfected with one or more recombinant nucleic acids that are provided as one or more plasmids. In some embodiments, the HEK293 cells are transfected with (i) one plasmid comprising a gene of interest flanked by ITRs, nucleic acid sequences for encoding AAV Rep and Cap proteins, and (ii) a second plasmid comprising nucleic acid sequences encoding AAV helper functions, such as a pAdhelper plasmid comprising nucleic acid sequences for helper genes e.g., E1A, E1B, E2A, VA, and E4orf6 functions. In some embodiments, the HEK293 cells are transfected with (i) one plasmid comprising a gene of interest flanked by ITRs, (ii) a second plasmid comprising nucleic acid sequences for encoding AAV Rep and Cap proteins, and (iii) a third plasmid comprising nucleic acid sequences encoding AAV helper functions, such as a pAdhelper plasmid comprising nucleic acid sequences for helper genes e.g., E1A, E1B, E2A, VA, and E4orf6 functions. In some embodiments, the one or more recombinant nucleic acids encode a recombinant AAV (rAAV) genome. In some embodiments, the rAAV genome comprises a gene of interest flanked by inverted terminal repeats (ITRs). In some embodiments, the gene of interest encodes an antibody, enzyme, growth factor, or hormone. In some embodiments, the ITRs comprise AAV2 ITRs or AAV9 ITRs. In some embodiments, the one or more recombinant nucleic acids encode a capsid (Cap) protein. In some embodiments, the capsid protein is an AAV1, AAV2, AAV3, AAV4, AAV5, AAV6, AAV7, AAV8, AAV9, or AAV10 capsid protein, or a variant, a chimera, or hybrid thereof. In some embodiments, the one or more recombinant nucleic acids also encode a Rep protein (e.g., a Rep protein from a suitable AAV serotype).

In some embodiments, the cell culture comprises producer cells. In some embodiments, the producer cell comprises stably integrated nucleic acid sequences that encode a heterologous gene of interest flanked by inverted terminal repeats (ITRs) and AAV Rep and Cap proteins. In some embodiments, one or more helper functions are provided to generate rAAV particles from the producer cells. In some embodiments, the one or more helper functions are provided by infecting the producer cells with a helper virus, such as an adenovirus or a herpes simplex virus (HSV). In some embodiments the helper virus encode AAV helper virus genes, e.g., E1A, E1B, E2A, VA, and E4orf6 functions. In some embodiments, the one or more helper functions are provided as a virus, optionally an Ad5 virus, optionally a wild-type Ad5 virus.

In some aspects, the present disclosure provides a method comprising a) incubating one or more recombinant nucleic acids and one or more transfection reagents in a first medium under conditions sufficient for complex formation; and b) transferring a volume of the first medium to a second medium, wherein the volume is at least 25 L. In some embodiments, the volume of the first medium is transferred to a second medium using a pump. In some embodiments, the pump is a pump that produces a low amount of sheer. In some embodiments, the pump is a low shear pump, such as a bioprocessing pump (e.g., a pump sold under the trademark Levitronix® and/or PuraLev®100).

In some embodiments, the volume of the first medium is transferred to a second medium using pressure, wherein the volume is at least 25 L, and wherein the pressure is greater than one atmosphere (atm). In some embodiments, the pressure is provided by a pump. In some embodiments, the pressure is greater than 1.5 atmospheres (atm) and less than 5 atm. In some embodiments, the pressure is about 2 atm. In some embodiments, the pressure is about 2.5 atm. In some embodiments, complex formation (step (a)) and a transfer step (step (b)) combined last 5-20 minutes (for example 8-12 minutes, 9-11 minutes, e.g., about 10 minutes).

In some embodiments, the first medium is the same as the second medium. In some embodiments, the first medium is different from the second medium. In some embodiments, the second medium further comprises a cell culture. In some embodiments, the cell culture comprises mammalian cells. In some embodiments, the cell culture comprises HEK cells, CHO cells, or HeLa cells. In some embodiments, the cell culture comprises HEK 293 cells.

In some embodiments, the one or more recombinant nucleic acids involved in complex formation comprise plasmids. In some embodiments, one of the one or more complexed recombinant nucleic acids encode a recombinant AAV (rAAV) genome. In some embodiments, the rAAV genome comprises a gene of interest flanked by inverted terminal repeats (ITRs). In some embodiments, the gene of interest encodes an antibody, a protein, an enzyme, a growth factor, a miRNA, or a hormone. In some embodiments, the ITRs comprise AAV2 ITRs. In some embodiments, one of the one or more complexed recombinant nucleic acids encode a capsid (Cap) protein. In some embodiments, the capsid protein is an AAV1, AAV2, AAV3, AAV4, AAV5, AAV6, AAV7, AAV8, AAV9, or AAV10 capsid protein, or a variant thereof. In some embodiments, one of the one or more complexed recombinant nucleic acids also encode a Rep protein. In some embodiments, the Cap and Rep proteins are encoded on a different recombinant nucleic acid than the recombinant nucleic acid encoding the rAAV genome.

In some embodiments, the cell culture of the transfer step (b) comprises producer cells. In some embodiments, one or more helper functions are provided. In some embodiments, the one or more helper functions are encoded on the one or more recombinant nucleic acids. In some embodiments, the one or more helper functions are provided as a virus, optionally an Ad5 virus.

In some aspects, the present disclosure contemplates a method of transferring a plurality of transfection complexes to a bioreactor, wherein the plurality of complexes are transferred as a single volume, and wherein single volume comprising the plurality of transfection complexes comprises a volume of at least 25L. In some embodiments, the capacity of the bioreactor is 25-50 L, 50 L-100 L, 100 L-500 L, 500 L-1,000 L, 1,000 L-5,000 L, or 5,000 L-10,000 L. In some embodiments, the plurality of complexes are transferred using a pressure greater than 1 atmosphere (atm). In some embodiments, wherein the pressure is provided by a pump. In some embodiments, the pressure is greater than 1.5 atm and less than 5 atm. In some embodiments, the pressure is about 2 atm. In some embodiments, the pressure is about 2.5 atm. 25 In some embodiments, the method of transferring a plurality of transfection complexes takes place in about 10 minutes.

In some embodiments, the plurality of transfection complexes comprises one or more nucleic acids and one or more transfection reagents. In some embodiments, the one or more nucleic acids comprises one or more plasmids. In some embodiments, one of the one or more plasmids comprised in the plurality of transfection complexes comprises elements sufficient for AAV production in a cell. In some embodiments, the one or more recombinant nucleic acids encode a recombinant AAV (rAAV) genome. In some embodiments, the rAAV genome comprises a gene of interest flanked by inverted terminal repeats (ITRs). In some embodiments, the gene of interest encodes an antibody, a protein, an enzyme, a growth factor, a miRNA, or a hormone. In some embodiments, the ITRs comprise AAV2 ITRs. In some embodiments, one of the one or more recombinant nucleic acids comprised in the plurality of transfection complexes encode a capsid (Cap) protein. In some embodiments, the capsid protein is an AAV1, AAV2, AAV3, AAV4, AAV5, AAV6, AAV7, AAV8, AAV9, or AAV10 capsid protein, or a variant, chimera, or hybrid thereof. In some embodiments, one of the one or more recombinant nucleic acids comprised in the plurality of transfection complexes also encodes a Rep protein.

Aspects of the present disclosure provide a method comprising:
a) incubating one or more recombinant nucleic acids and one or more transfection reagents in a vessel in a first medium under conditions sufficient for complex formation; and
b) contacting a cell culture with a volume of the first medium, wherein the volume is at least 25 L;
a) incubating the cell culture for 4-8 hours under conditions sufficient for nucleic acid transfection; and
d) contacting the cell culture of c) with a second medium.

In some embodiments, step (a) produces a plurality of transfection complexes, and wherein the plurality of complexes is transferred to a bioreactor comprising the cell culture as a single volume.

### BRIEF DESCRIPTION OF DRAWINGS

FIG. 1 illustrates a non-limiting embodiment of a process 100 for transfection complex formation.

FIG. 2 illustrates a non-limiting embodiment of a process 200 for improving virus production by a cell culture.

FIG. 3 shows an overview of the current method of transferring transfection complexes to a bioreactor (top) and a non-limiting example of an improved method (bottom).

FIG. 4 shows a previous scale up transfection method suitable for use with a 500 L or larger bioreactor comprising a HEK293 cell culture.

FIG. 5 shows a non-limiting example of an updated method for scaling up a transfection using pressure. This method is suitable for use with a 500 L or larger bioreactor comprising a mammalian cell culture (e.g., HEK293) cell culture and presents fewer opportunities for contamination.

FIGS. 6A, 6B and 6C show graphs of viral genome titers harvested from cells transfected using transfection complexes generated using a prior method (control) or the presently-disclosed method. Across multiple replicates, the presently-disclosed method performs as well as a current method. As shown in FIG. 6A, the control is a shake flask transfection, LevMixer® 1, 2, 3, 4 show results of independent shake flask replicates combined with LevMixer® complex formation. As shown in FIG. 6B, the control is a shake flask transfection, LevMixer® 1 and 2 show results of shake flasks infected with LevMixer®-formed complex in small scale proof of concept.

As shown in FIG. 6C, LevMixer® complex showing cells transfected with the complex that was formed in the LevMixer® were compared with control transfections using conventional transfection methods.

FIGS. 7A, 7B, 7C, and-7D show the scalability of the present methods. FIG. 7A shows a diagram depicting scaling up manufacturing from 250 mL reactors to 5 L reactors, 50 L reactors, and then to 500 L bioreactors. FIG. 7B shows the increase in productivity relative to a current method (“conventional”) resulting from combining the presently-disclosed methods with a dual plasmid system (“+dual”), improved feeding method (“feed”), or both (“+dual+feed”). FIG. 7C shows the relative genome titer achieved by using the presently-described method in 250 ml reactors, 3L reactors, 5L reactors, 50 L reactors, or 500 L bioreactors. Values are normalized to the genome titer observed at 500 L. FIG. 7D shows the relative capsid titer achieved by using the presently-described method in 250 mL reactors, 3L reactors, 5L reactors, 50 L reactors, or 500 L bioreactors. Values are normalized to the capsid titer observed at 500 L.

FIGS. 8A-8B illustrate non-limiting examples of the steps of the method described in this application. In some embodiments, the steps of FIG. 8A occur within acts 205 through 210 of FIG. 2.

FIG. 9 depicts non-limiting examples of a transfection process. In the top panel, three plasmids (Ad Helper, RepCap and Transgene flanked by ITRs) are transfected with the transfecting agent (TA) into HEK293 cells. In the lower panel, two plasmids (Ad Helper and a second plasmid comprising RepCap and Transgene flanked by ITRs) are transfected with the transfecting agent (TA) into HEK293 cells. In some embodiments, the respective genes are expressed and help in the replication and packaging of the AAV.

FIG. 10 shows a non-limiting exemplary method for preparing, transfecting, maintaining, and harvesting rAAV from HEK293 cells.

FIG. 11 shows an embodiment of the presently disclosed method. In this embodiment, adding a second medium (as first feed) occurs between 4 and 8 hours post transfection (PT) and a second feed occurs at 24 hours post transfection. In this embodiment, rAAV is isolated and/or the cells are harvested about 48 hours post-transfection.

FIGS. 12A-12B show the increase in viral titer when a non-limiting embodiment of the presently-described method is used. FIG. 12A shows a diagram comparing the steps of the conventional method and a non-limiting example of the presently-described method. FIG. 12B shows fold change in productivity when the feed strategy of the application is applied to triple transfection plasmid system (3 plasmids). This graph is plotted by taking average fold change between technical replicate experiments. The left bar shows the result using the triple transfection process (3 plasmids) with a conventional feed strategy (“conventional feed”). The right bar shows the result using the triple transfection process (3 plasmids) with a feed strategy of the application (“new feed”).

FIGS. 13A-13B show improvement in viral particle production using an embodiment of the presently disclosed methods compared to the method of FIG. 10. The improvement was observed both with a triple plasmid system (FIG. 13A) and a dual plasmid system (FIG. 13B).

FIGS. 14A, 14B and, 14C show the evaluation of media feed timing on viral particle production. Feeding the cells at 4 hours pre-transfection was found to be detrimental. Feeding the cells 4 hours post transfection improved titers by 3-fold when compared to control. Total capsids were found to be similar to the control. Percent full capsids were found to be 3-fold than the control.

FIG. 15 shows the fold change in productivity (about 2.8 fold) when a dual transfection plasmid system (2 plasmids) is used (right bar, “double”) compared to a triple transfection plasmid system (3 plasmids) (left bar, “triple”), in combination with the methods of the disclosure.

FIGS. 16A, 16B, and 16C show the significant improvement in viral particle productivity and product quality. Reproducible titers were observed for the control on D3 (48 hours post-transfection) and D4 (Day 4, 72 hours post-transfection). Feeding the cells 4 hours post transfection improved titers by 2-fold when compared to control on D4. 4 hour-PT feed shows maximum productivity on D3, unlike the control. Extending the feed factor (over feeding) did not impact the productivity. Total capsids on D4 were found to be higher than D3, which significantly impacted the product quality. Overall productivity was high on D3, in terms of both productivity (titer) and product quality (percent full capsids). The 4 hour-PT feed shows potential to shorten the process by one day.

FIG. 17 shows the media feed at 4-8 hours PT, and its benefit to AAV productivity. AAV productivity peaked when cells were fed at 8 hours PT and D2 (24 hours post-transfection). D1 feed timing ranged from 4-8 hours PT, with a decrease in productivity observed after 4-8 hours PT. Feeding only D2 was not as beneficial, which indicated the importance of a 4-hour PT feed. Glucose only or media minus glucose was not as beneficial, which indicates that media as a whole is important for AAV productivity.

FIGS. 18A, 18B, and 18C show the impact of feeding on day 2 (D2) (24 hours post-transfection) compared to day 3 (D3) feed. FIG. 18A shows the titer of viral genomes measured after harvest for each tested condition. FIG. 18B shows the total number of capsids harvested for each tested condition. FIG. 18C shows the percentage of harvested capsids that contained viral vector (“full capsids”) for each tested condition. Results show that feeding 4 h post transfection (on day 1 (D1)) and on D2 is sufficient for the overall productivity. Feeding on D3 does not appear to improve harvest, and thus harvesting may be done earlier than expected (on D3 rather than D4).

### DETAILED DESCRIPTION

Aspects of the present disclosure provide improved methods for culturing cells for viral production. In some aspects, the methods are useful for rAAV production. In some aspects, the methods are useful for increasing total viral production by cells. In some aspects, the methods are useful for increasing the percentage of full viral capsids produced. In some embodiments, the methods are useful for decreasing the time needed to harvest the virus. In some aspects, the present disclosure relates to providing media (e.g., a feed medium) to cells. In some aspects, the present disclosure contemplates providing a second medium (e.g., a feed medium) to cells after the cells are incubated under conditions sufficient for nucleic acid transfection in a first medium. In some embodiments, the first medium comprises a production media, (e.g., media sold under the trademark LV-MAX™). In some embodiments, the second medium is provided after a set incubation time post-transfection. In some embodiments, the second medium is provided after 1-20 hours (e.g., 2-15 hours, for example 3-10 hours or 4-8 hours) of incubation post-transfection. In some embodiments, the second medium is provided when a metabolite meets or exceeds a threshold concentration in the first medium. In some embodiments, the second medium is provided when a nutrient falls at or below a threshold level in the first medium. In some embodiments, productivity is increased by 2-3 fold. In some embodiments, further method updates lead to a 10-fold increase in productivity. In some embodiments, an increase in productivity indicates an increase in viral genomes measured after harvest. In some embodiments, an increase in productivity indicates an increase in viral capsids harvested. In some embodiments, an increase in productivity indicates an increase in the percentage of full capsids harvested.

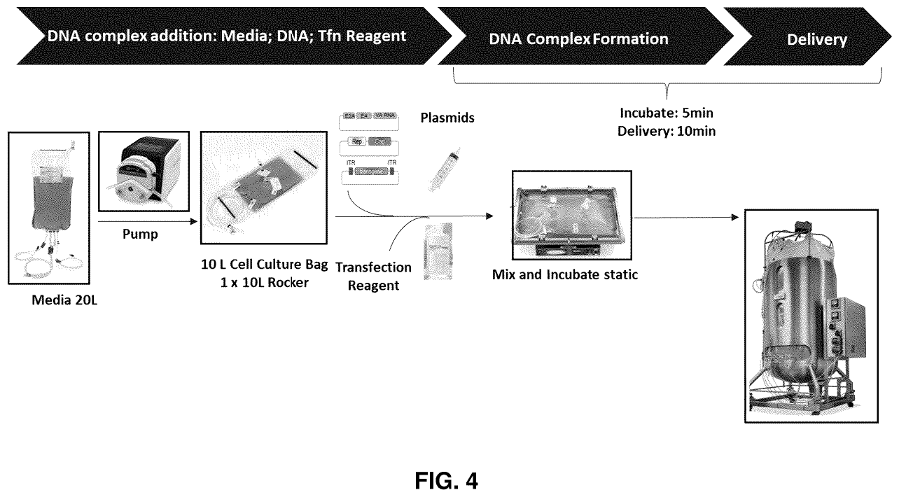

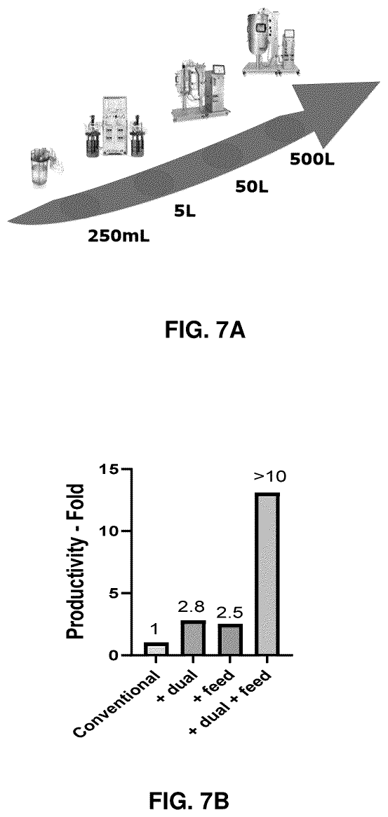

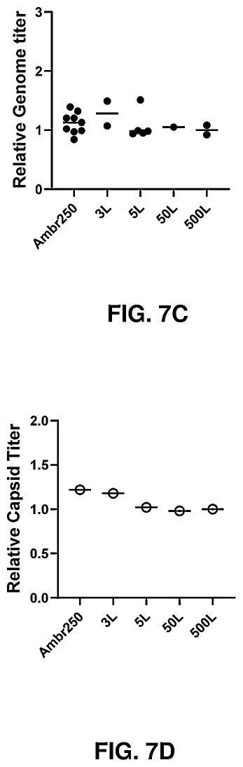

Large volumes complicate the rAAV particle production process because viral (e.g., rAAV) production is time-sensitive and combining small batches increases the likelihood of contamination. An example of a standard protocol is shown in FIG. 4. Such protocols are difficult to perform on a large scale due to limitations in mixing elements to form transfection complexes and transferring formed transfection complexes to a bioreactor. By contrast, in some embodiments, successful scale-up of the entire manufacturing process using the methods and compositions associated with the present disclosure (as shown in FIG. 7A) is demonstrated from 250 mL reactors to 500 L reactors.

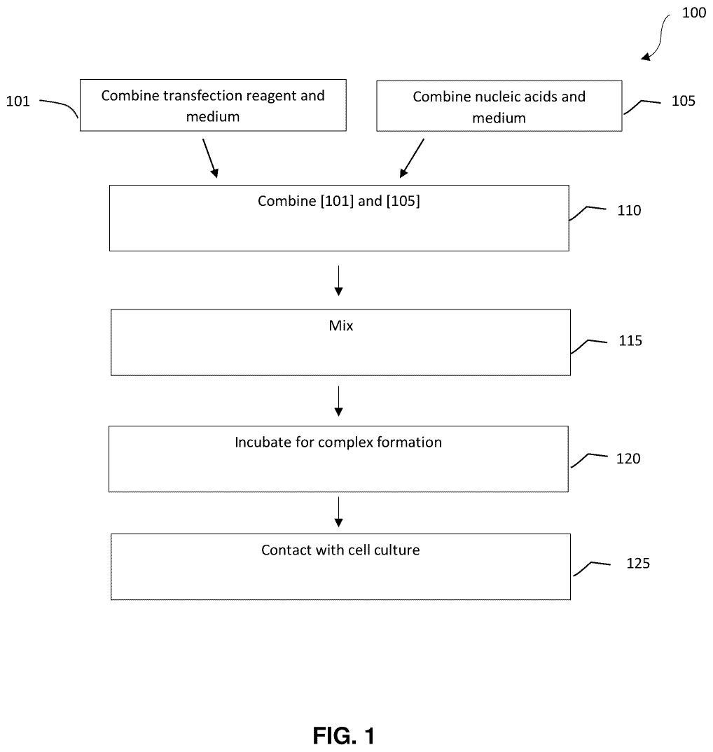

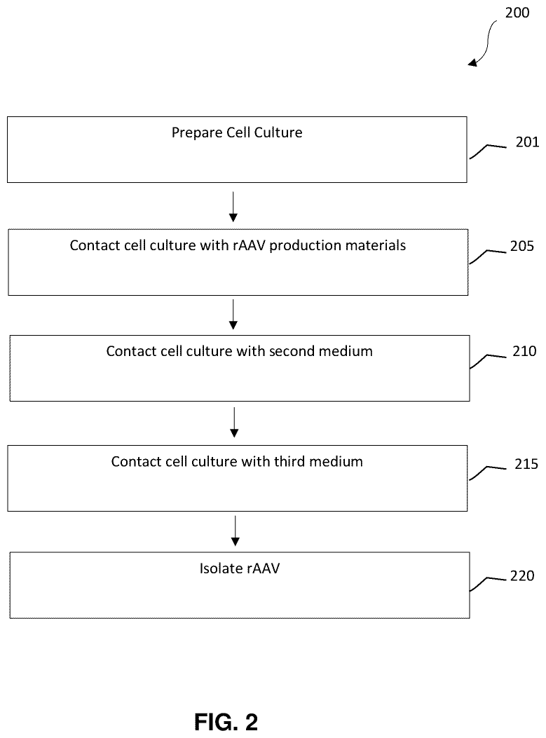

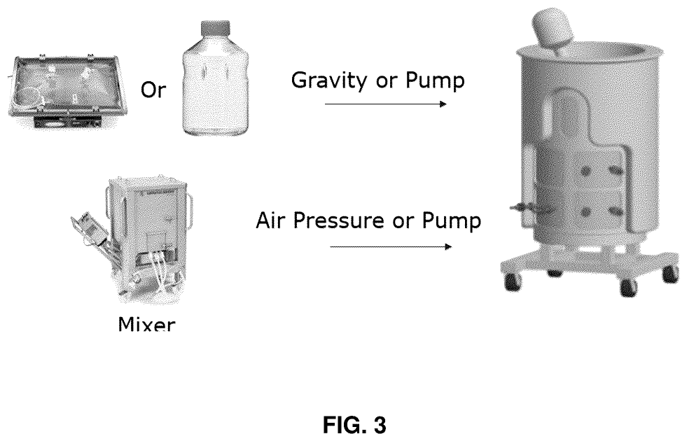

Aspects of the application relate to processes for mixing elements to form transfection complexes and processes for transferring formed transfection complexes to a bioreactor. Non-limiting examples of processes that can be performed on a large scale (e.g., in 25-500 L or larger volumes) are provided in FIGS. 1 and 2.

FIG. 1 illustrates a non-limiting embodiment of a process 100 for transfection complex formation. In act 101, one or more transfection reagents are combined with medium. In act 105, one or more nucleic acids are combined with medium. In act 110, the combinations of act 101 and act 105 are combined. In act 115, the combination of act 110 is mixed. In act 120, the mixture of step 115 is incubated for complex formation. In act 125, the result of step 120 is contacted with a cell culture.

In some embodiments, the method of FIG. 1 is used to form transfection complexes. In some embodiments, the method of FIG. 1 is useful for scaling up production of rAAV. In some embodiments, one or more of the acts illustrated in FIG. 1 can be omitted and/or combined with other acts of FIG. 1. In some embodiments, act 110 and act 115 may be combined.

In some embodiments, one or more of the acts illustrated in FIG. 1 may be combined with one or more acts illustrated in FIG. 2. For example, act 125 of FIG. 1 may comprise contact with the cell culture of act 201, wherein the complexes of act 125 comprise the rAAV production materials of act 205.

In some embodiments, act 101 comprises combining one or more transfection reagents with medium, for example, using any suitable technique for combining solutions and/or adding dry material to a solution, e.g., using a pipettor or other suitable device for handling solutions.

In some embodiments, a transfection reagent comprises a commercially available transfection reagent. In some embodiments, a transfection reagent comprises a chemical transfection reagent. In some embodiments, a transfection reagent comprises a liposomal-based transfection reagent. In some embodiments, a transfection reagent comprises a liposome. In some embodiments, the liposome is a positively charged or cationic liposome. In some embodiments, a transfection reagent comprises a non-liposomal-based transfection reagent. In some embodiments a transfection reagent comprises a calcium phosphate, dendrimer, polymer, nanoparticle, or non-liposomal lipid. In some embodiments, a transfection reagent comprises lipofectamine or a variant thereof. In some embodiments, transfection is carried out by electroporation nucleofection which is a method that uses a combination of electrical and chemical factors, using a device (e.g., Nucleofector™ by Lonza). Transfection reagents include, but are not limited to, polyethylenimine (PEI), animal-free transfection reagents (e.g., such as those sold under the trademark FectoVIR® (polyplus)), pure lipid or high-lipid based transfection agents (such as those sold as: Nanofectamine (GE Healthcare), Oligofectamine (Invitrogen), RNAiMAX (Invitrogen), siPORT (ThermoFisher), DharmaFECT (Dharmacon), Endofectin@MAX (GeneCopocia), Escort IV Liposome (Sigma-Aldrich)), and mixed lipid and/or non-lipid based transfection reagents (such as those sold as Arrest-In (Dharmacon), TurboFect (Thermo), Effectene (Qiagen), Attractene (Qiagen), PolyFect (Qiagen), SuperFect (Qiagen), ExpressFect (Thomas), GeneJammer (Stratagene), FuGENE (Promega), INTERFERin (Polyplus), NanoFectin (System Biosciences), X-tremeGENE (Roche), Xfect (ClonTech), Escort IV (Sigma-Aldrich), and N-TER (Sigma-Aldrich)).

In some embodiments, a medium (e.g., a first, a second, or a third medium) is an appropriate medium for cell growth and/or transfection. In some embodiments, the medium is a medium that supports cell growth. In some embodiments, the medium provides one or more nutrients (e.g., trace metal, vitamin, carbon source, amino acid source, or antioxidant source). In some embodiments, a carbon source is glucose or galactose. In some embodiments, a carbon source is provided at 50-200 grams/L of medium. In some embodiments, a carbon source is provided at 25 g/L, 30 g/L, 35 g/L, 40 g/L, 45 g/L, 50 g/L, 55 g/L, 60 g/L, 65 g/L, 70 g/L, 75 g/L, 80 g/L; 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, 150 g/L, 155 g/L, 160 g/L, 165 g/L, 170 g/L, 175 g/L, 180 g/L; 185 g/L, 190 g/L, 195 g/L, 200 g/L, 205 g/L, 210 g/L, 215 g/L, or 220 g/L. In some embodiments, at least one medium is a feed medium. In some embodiments, one or more of the media comprise a complex medium. In some embodiments, the medium is a defined medium. In some embodiments, one or more of the media comprise Minimal Essential Medium (MEM), Eagle's Minimum Essential Medium (EMEM), Dulbecco's Modified Eagle's Medium (DMEM), LV-MAX™ media (Gibco), EX-Cell media, or RPMI media. In some embodiments, the medium is a serum-free medium. In some embodiments, the medium is supplemented with serum. In some embodiments, the medium is an appropriate medium for growth and/or transfection of mammalian cells. In some embodiments, the medium is an appropriate medium for growth and/or transfection of HEK293 cells. In some embodiments, the medium is an appropriate medium for growth and/or transfection of HeLa cells. In some embodiments, the medium is an appropriate medium for AAV production. In some embodiments, a second medium further comprises one or more additional nutrients (e.g., carbon source, amino acid source, trace metal, antioxidant source, and/or vitamin) relative to a first medium. In some embodiments, a third medium further comprises one or more additional nutrients (e.g., carbon source, amino acid source, trace metal, antioxidant source, and/or vitamin) relative to a first and/or a second medium.

In some embodiments, act 105 comprises combining one or more nucleic acids with medium. In some embodiments, the one or more nucleic acids are recombinant nucleic acids. In some embodiments, the one or more recombinant nucleic acids comprise deoxyribonucleic acid (DNA) in some embodiments, the one or more recombinant nucleic acids comprise ribonucleic acid (RNA).

In some embodiments, the one or more recombinant nucleic acids comprise one or more plasmids. In some embodiments, the one or more recombinant nucleic acids are provided as one or more plasmids. In some embodiments, the plasmids comprise further polynucleotide sequences, such as one or more replication elements or regulatory elements, such as promoters and/or transcriptional control elements. In some embodiments, the one or more nucleic acids comprise two plasmids. In some embodiments, the two plasmids comprise a dual transfection system. In some embodiments, the one or more nucleic acids comprise three plasmids. In some embodiments, the three plasmids comprise a triple transfection system. In some embodiments, multiple molar ratios of plasmids are studied. In some embodiments, in the dual transfection system, one plasmid comprises a gene of interest flanked by ITRs, nucleic acid sequences for encoding AAV Rep and Cap proteins, and a second plasmid comprises nucleic acid sequences encoding AAV helper functions, such as a pAdhelper plasmid comprising nucleic acid sequences for helper genes e.g., E1A, E1B, E2A, VA, and E4orf6 functions. In some embodiments, switching from a three plasmid system (also referred to herein as a “triple transfection plasmid system”) to a dual plasmid system increases AAV particle production by at least 2 fold. In some embodiments, AAV particle production is increased by about 3 fold.

In some embodiments, the one or more recombinant nucleic acids encode a recombinant AAV (rAAV) genome. In some embodiments, the rAAV genome comprises a gene of interest flanked by inverted terminal repeats (ITRs). In some embodiments, the ITRs comprise AAV2 ITRs or AAV9 ITRs. In some embodiments, the gene of interest encodes an antibody, enzyme, protein, growth factor, miRNA, or hormone. In some embodiments, the one or more recombinant nucleic acids encode a capsid (Cap) protein. In some embodiments, the capsid protein is an AAV1, AAV2, AAV3, AAV4, AAV5, AAV6, AAV7, AAV8, AAV9, AAV10, AAV11, or AAV12 capsid protein, or a variant thereof. In some embodiments, the one or more recombinant nucleic acids also encode a Rep protein.

In some embodiments, the medium used in act 101 and/or act 105 is a medium as described above. In some embodiments, the medium in act 101 and act 105 are the same medium. In some embodiments, the medium in act 101 and act 105 are different media.

In some embodiments, act 101 and act 105 each occur in a vessel. In some embodiments, a vessel comprises a plastic, polymeric, glass, or metal vessel. In some embodiments, a vessel is a single use vessel. In some embodiments, act 101 and/or act 105 each occur in a bottle. In some embodiments, act 101 and/or act 105 cach occur in a bag.

In some embodiments, act 110 comprises combining the mixtures of act 101 and act 105. In some embodiments, act 110 comprises transferring the mixtures of act 101 and act 105 to a different vessel (e.g., a subsequent vessel). In some embodiments, the subsequent vessel comprises a plastic, polymeric, glass, or metal vessel. In some embodiments, the subsequent vessel is a single use vessel. In some embodiments, the vessel of act 110 is comprised in an apparatus capable of mixing. In some embodiments, the vessel of act 110 is comprised in an apparatus capable of gentle mixing, such as a bag. In some embodiments, the vessel of act 110 is comprised in a mixer. In some embodiments, the mixer comprises an impeller. In some embodiments, the mixer comprises a magnetic or levitating impeller (e.g., a mixer sold under the trademark LevMixer®). In some embodiments, act 110 and act 115 occur simultaneously.

In some embodiments, act 115 comprises mixing the combination of act 110. In some embodiments, act 115 comprises gently mixing the combination of act 110. In some embodiments, gently mixing comprises using a mixer with a low shear rate (“y”). In some embodiments, a low shear rate is a shear rate that is not detrimental to transfection complexes. In some embodiments, low shear comprises shear that is less than 2650 y, less than 2600 y, less than 2550 y, less than 2500 y, less than 2450 y, less than 2400y, less than 2300 y, less than 2250 y, less than 2200 y, less than 2150 y, less than 2100 y, less than 2050 y, less than 2000 y, less than 1950 y, less than 1900 y, less than 1850 y, or less than 1800 y. In some embodiments, gentle mixing is performed by a mixer comprising an impeller. In some embodiments, gentle mixing is performed using a mixer comprising a magnetic or levitating impeller (e.g., a mixer sold under the trademark LevMixe®). In some embodiments, formed transfection complexes are transferred to a subsequent vessel using tubing with a low shear rate. In some embodiments, shear rate is calculated as y=4q/πR³, where “y” is the shear rate/second, “q” is the volume flow through the pipe, cm³/s, and “R” is the pipe radius.

In some embodiments, act 120 comprises incubating the mixture of act 115 for complex formation. In some embodiments, act 120 comprises incubating the mixture without mixing. In some embodiments, act 120 comprises incubating the mixture with gentle mixing. In some embodiments, act 120 and act 125 occur within about 10 minutes. In some embodiments, act 120 and act 125 occur within 10-20 minutes, 5-15 minutes, 8-12 minutes, 9-13 minutes, 7-10 minutes, or 9-11 minutes. In some embodiments, act 120 and act 125 occur within about 5 minutes, about 10 minutes, about 15 minutes, about 20 minutes, or about 25 minutes.

In some embodiments, act 125 comprises contacting the incubated mixture of act 120 with a cell culture. In some embodiments, the vessel of act 115 and/or act 120 is connected to a bioreactor via tubing, piping, or other fluid connector. In some embodiments, the tubing, piping or other fluid connector comprises a standard-sized tubing, piping, or fluid connector (e.g., with an inner diameter of 1-25 mm, for example 2-15 mm (e.g., 3.18 mm-12.7 mm)). In some embodiments, tubing, piping, or other fluid connector is made of plastic, polymeric material, glass, or metal. In some embodiments, the tubing piping, or other fluid connector is manufactured tubing (e.g., sold under the trademark Masterflex® or C-flex®). In some embodiments, pressure is applied to the vessel to transfer medium to the bioreactor via the fluid connector. In some embodiments, medium is pumped through the fluid connector from the vessel into the bioreactor. In some aspects, the present disclosure relates to scaled-up methods. In some embodiments, the volume of the incubated mixture of act 120 is at least 25 L. In some embodiments, the volume of the incubated mixture of act 120 is 25 L, 30 L, 35 L, 40 L, 45 L, 50 L, 55 L, 60 L, 65 L, 70 L, 75 L, 80 L, 85 L, 90 L, 95 L, 100 L, 110 L, 120 L, 130 L, 140 L, 150 L, 160 L, 170 L, 180 L, 190 L, 200 L, 210 L, or 220 L. In some embodiments, the volume of the incubated mixture of act 120 is about 10 percent of the volume of the bioreactor.

In some embodiments, act 125 comprises transferring a plurality of transfection complexes to a bioreactor, wherein the plurality of complexes is transferred as a single volume. In some aspects, the present disclosure relates to scaled-up methods. In some embodiments, the single volume is at least 25 L. In some embodiments, the single volume is about 25 L, about 30 L, about 35 L, about 40 L, about 45 L, about 50 L, about 55 L, about 60 L, about 65 L, about 70 L, about 75 L, about 80 L, about 85 L, about 90 L, about 95 L, about 100 L, about 110 L, about 120 L, about 130 L, about 140 L, about 150 L, about 160 L, about 170 L, about 180 L, about 190 L, about 200 L, about 210 L, or about 220 L. In some embodiments, the single volume is about 10 percent the volume of the bioreactor.

In some embodiments, the vessel of act 115 and/or act 120 comprises 10 percent of the volume of bioreactor of act 125. For example, the vessel of act 115 and/or act 120 comprises a volume of 50 L and the bioreactor of act 125 comprises a volume of 500 L.

In some aspects, the methods are useful for expediting transfer of transfection components from a vessel to a bioreactor, as set forth in act 125. In some embodiments, transferring the volume of first medium comprising the transfection reagent and nucleic acids or the plurality of transfection complexes in a short time is important for the success of the method. In some embodiments, the transfer takes place in about 5 minutes, about 10 minutes, about 15 minutes, about 20 minutes, or about 25 minutes. In some embodiments, the transfer takes place in about 10-20 minutes, 5-15 minutes, 8-12 minutes, 9-13 minutes, 7-10 minutes, or 9-11 minutes. In some embodiments, the transfer takes place in about 10 minutes.

In some aspects, the methods are useful for reducing opportunities for contamination. Currently available alternatives involve transferring multiple smaller batches of transfection components to a bioreactor. In some embodiments, multiple transfers introduce additional opportunities for contamination. In some embodiments, transferring the transfection reagent and nucleic acids or plurality of transfection complexes as a single volume reduces the likelihood of contamination. In some embodiments, the methods are useful for simplifying the manufacturing process. In some embodiments, conventional methods of transfection are cumbersome and non-scalable. In some embodiments, the method described in this application is less cumbersome and more scalable relative to conventional methods.

In some aspects, the presently disclosed methods comprise transferring the transfection reagent and nucleic acids or plurality of transfection complexes using pressure. In some embodiments, the pressure is at least 1 atmosphere (atm). In some embodiments, the pressure is about 1.5 atm, about 2 atm, about 2.5 atm, about 3 atm, about 3.5 atm, about 4 atm, about 4.5 atm, about 5 atm, about 5.5 atm, or about 6 atm. In some embodiments, the pressure is between 1.5 atm and 5 atm. In some embodiments, the pressure is between 1 atm and 4 atm. In some embodiments, the pressure is between 2 atm and 4 atm. In some embodiments, the pressure is between 2 atm and 3 atm.

In some embodiments, the pressure is air pressure. In some embodiments, the pressure is provided by a pump. In some embodiments, the pump is a low shear pump, such as a bioprocessing pump (e.g., a pump sold under the trademark Levitronix® and/or PuraLev®100). In some embodiments, the pump comprises a magnetic or levitating impeller. In some embodiments, the pump is part of a mixing apparatus. In some embodiments, the mixing apparatus comprises a vessel. In some embodiments, the pump is part of a mixer comprising a levitating or magnetic impeller (e.g., a mixer sold under the trademark LevMixer®), or other apparatus capable of gentle mixing.

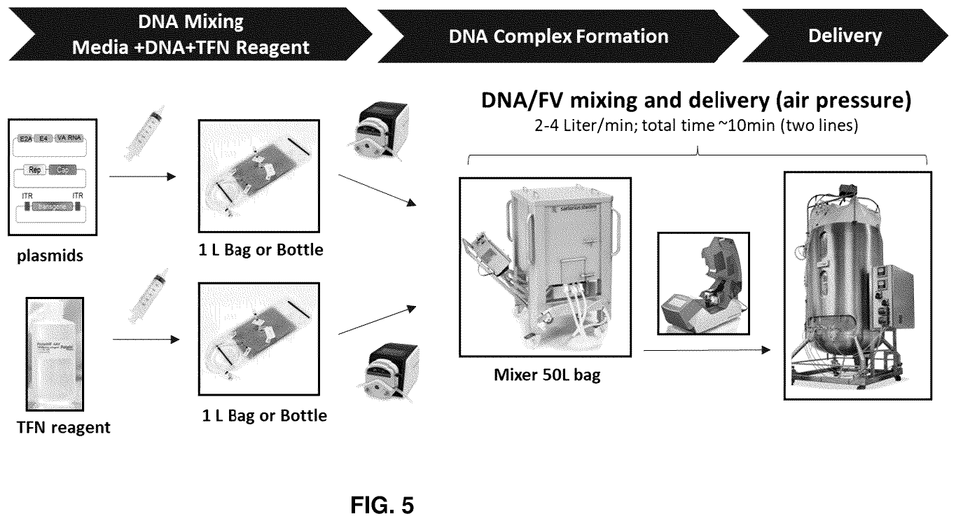

A non-limiting embodiment of process 100 is shown in FIG. 5.

In some embodiments, the cell culture is the cell culture of act 201. In some embodiments, act 125 comprises contacting the incubated mixture of act 115 with the cells of at 201 according to act 205.

In some embodiments, following act 125, the process further comprises producing rAAV particles. In some embodiments, rAAV particles are isolated and prepared for administration to a subject in need of treatment.

FIG. 2 illustrates a non-limiting embodiment of a process 200 for improving virus production by a cell culture. In act 201, a cell culture is prepared. In act 205, the cell culture is contacted with rAAV production materials (e.g., nucleic acids encoding one or more rAAV genomes or proteins and/or one or more helper functions and/or one or more helper viruses). In act 210, the cell culture is contacted with a second medium (e.g., a feed medium). In act 215, the cell culture is contacted with a third medium. In act 220, rAAV is isolated from the cell culture.

In some embodiments, the method of FIG. 2 is used to improve virus production by a cell culture. In some embodiments, one or more of the acts illustrated in FIG. 2 can be omitted, or alternatively repeated two or more times, and/or combined with other acts. Non-limiting examples of one or more acts that can be omitted include act 215 and act 220.

In some aspects, the process of FIG. 2 is combined with the process of FIG. 1, as described above. However, the process of FIG. 2 may be used with any rAAV production materials. The process of FIG. 1 may be used with any cell growth and/or rAAV production method, including, but not limited to the process of FIG. 2.

In some embodiments, act 201 comprises preparing a cell culture. In some embodiments, preparing a cell culture comprises inoculating a cell culture and/or growing the cell culture to an appropriate density. In some embodiments, act 201 comprises preparing a cell culture for infection and/or transfection. In some embodiments, act 201 comprises preparing the cell culture in a first medium (e.g., growing the cell culture in a medium). Many cell types may be cultured in vitro. In some embodiments, the cell culture comprises animal cells. In some embodiments, the cell culture comprises mammalian cells. In some embodiments, the cell culture comprises human cells. In some embodiments, the cell culture comprises epithelial cells. In some embodiments, the cell culture comprises HEK cells, CHO cells, or HeLa cells. In some embodiments, the cell culture comprises HEK293 cells. In some embodiments, the cell culture comprises HeLa cells.

In some embodiments, the cell culture comprises producer cells. In some embodiments, a producer cell stably expresses rep and cap genes suitable for rAAV packaging. In some embodiments, a producer cell further comprises an rAAV genome. In some embodiments, the rAAV genome comprises a therapeutic gene.

In some embodiments, act 205 comprises contacting the cell culture with rAAV production materials.

In some embodiments, rAAV production materials comprise one or more recombinant nucleic acids. In some embodiments, the one or more recombinant nucleic acids comprise deoxyribonucleic acid (DNA) in some embodiments, the one or more recombinant nucleic acids comprise ribonucleic acid (RNA). In some embodiments, the one or more recombinant nucleic acids are delivered as part of the transfection complexes formed in process 100.

In some embodiments, the one or more recombinant nucleic acids comprise one or more plasmids. In some embodiments, the one or more nucleic acids comprise two plasmids. In some embodiments, the two plasmids comprise a dual transfection system. In some embodiments, the one or more nucleic acids comprise three plasmids. In some embodiments, the three plasmids comprise a triple transfection system. In some embodiments, multiple molar ratios of plasmids are studied. In some embodiments, switching from a three plasmid system (also referred to herein as a “triple transfection plasmid system”) to a dual plasmid system increases AAV particle production by at least 2 fold. In some embodiments, AAV particle production is increased by about 3 fold.

In some embodiments, the one or more recombinant nucleic acids encode a recombinant AAV (rAAV) genome. In some embodiments, the rAAV genome comprises a gene of interest flanked by inverted terminal repeats (ITRs). In some embodiments, the ITRs comprise AAV2 ITRs or AAV9 ITRs. In some embodiments, the gene of interest encodes an antibody, enzyme, protein, growth factor, miRNA, or hormone. In some embodiments, the one or more recombinant nucleic acids encode a capsid (Cap) protein. In some embodiments, the capsid protein is an AAV1, AAV2, AAV3, AAV4, AAV5, AAV6, AAV7, AAV8, AAV9, AAV10, AAV11, or AAV12 capsid protein, or a variant thereof. In some embodiments, the one or more recombinant nucleic acids also encode a Rep protein.

In some embodiments, the cell culture comprises producer cells and the rAAV production materials provide helper functions. In some embodiments, rAAV production materials comprise a helper virus. In some embodiments, the helper virus is adenovirus. In some embodiments, the helper virus is Ad5.

In some embodiments, act 205 occurs under conditions sufficient for nucleic acid transfection. In some embodiments, conditions sufficient for nucleotide transfection are conditions sufficient for successful transfection of more than 40%, more than 50%, more than 60%, more than 70%, more than 80% or more than 90% of the cells comprising the cell culture.

In some embodiments, act 205 occurs under conditions sufficient for infection with a helper virus. In some embodiments, conditions sufficient for infection with a helper virus are conditions sufficient for successful transfection of more than 40%, more than 50%, more than 60%, more than 70%, more than 80% or more than 90% of the cells comprising the cell culture.

In some embodiments, act 210 comprises contacting the cell culture with a second medium.

Aspects of the present disclosure provide methods of increasing rAAV production by contacting the cell culture with a second medium (e.g., a feed medium) after incubating the cell culture as described in act 205. In some embodiments, the second medium is added at a set time of incubation. In some embodiments, the second medium is added once one or more conditions are met. In some embodiments, feeding post-transfection is highly beneficial. In some embodiments, multiple time points are tested.

In some embodiments, act 210 occurs after a set incubation time. In some embodiments, act 210 occurs after 2-20 hours of incubation. In some embodiments, act 210 occurs after 2-16 hours of incubation. In some embodiments, act 210 occurs after 4-12 hours of incubation. In some embodiments, act 210 occurs after 4-16 hours of incubation. In some embodiments, act 210 occurs 2-12 hours of incubation. In some embodiments, act 210 occurs after 4-14 hours of incubation. In some embodiments, act 210 occurs after 4-8 hours of incubation. In some embodiments, act 210 occurs after 2-8 hours of incubation. In some embodiments, act 210 occurs after 2-6 hours of incubation. In some embodiments, act 210 occurs after 4-6 hours of incubation.

In some embodiments, act 210 is followed by act 215, e.g., after 24 hours of total incubation.

In some aspects, the present disclosure relates to methods of monitoring a cell culture to determine when to add the second medium (e.g., feed medium). In some embodiments, one or more characteristics of the cell culture are monitored. In some embodiments, the one or more characteristics of the cell culture comprises pH, CO2 emission, oxygen level, culture volume, lactate level, level of a nutrient, level of a metabolite, or carbon source level. In some embodiments, “monitoring” comprises taking one or more measurements during incubation. In some embodiments, oxygen demand is increased using a feed strategy of the application.

In some embodiments, the cell density of the cell culture is monitored. In some embodiments, the cell culture is contacted with the second medium (e.g., feed medium) when the cell culture reaches a cell density. In some embodiments, the cell culture is contacted with the second medium when the cell density is between about 1×10^6 and about 1×10^7. In some embodiments, the cell density is between about 2×10^6 and about 9×10^6, between about 3×10^6 and about 8×10^6, between about 4×10^6 and about 7×10^6, between about 2×10^6 and about 8×10^6, between about 2×10^6 and about 7×10^6, between about 2×10^6 and about 6×10^6, between about 2×10^6 and about 5×10^6 between about 3×10^6 and about 9×10^6, between about 3×10^6 and about 7×10^6 between about 3×10^6 and about 6×10^6, between about 4×10^6 and about 9×10^6, between about 4×10^6 and about 8×10^6, between about 4×10^6 and about 6×10^6, between about 5×10^6 and about 1×10^7, or between about 1×10^6 and about 5×10^6.

In some embodiments, the osmolarity of the culture medium is monitored. In some embodiments, the cell culture is contacted with the second medium when the osmolarity is between 260 and 320 milliosmoles (mOsm), between 270 and 310 mOsm, between 280 and 300 mOsm, between 265 and 280 mOsm, between 275 and 290 mOsm, between 285 and 300 mOsm, between 295 mOsm and 310 mOsm, or between 305 and 320 mOsm.

In some embodiments, pH of the medium is monitored. In some embodiments, the cell culture is contacted with the second medium when the pH is between 6.9 and 7.5, between 7 and 7.4, between 7.1 and 7.3, between 6.9 and 7.2, or between 7 and 7.3.

In some embodiments, viability of the cells comprising the cell culture is monitored. In some embodiments, the cell culture is contacted with the second medium when the viability of the cells is between about 90% and about 100%, between about 95% and about 100%, between about 96% and about 99%, between about 95 and about 99%, between about 95% and about 98%, or between about 95% and about 98.5%.

In some aspects, the present disclosure contemplates methods of monitoring a concentration of one or more molecules in the first medium and contacting the cell culture with a second medium (e.g., a feed medium) when the one or more molecules reaches a threshold value. In some embodiments, monitoring a concentration of one or more molecules comprises continuous monitoring. In some embodiments, monitoring a concentration of one or more molecules comprises taking periodic measurements to assess the concentration of one or more molecules. In some embodiments, the method further comprises contacting the cell culture with a third medium (e.g., a feed medium) after 24 hours of incubation, as in act 215.

In some embodiments, the second medium (e.g., a feed medium) is added when the concentration of a metabolite reaches or exceeds a threshold value. In some embodiments, a “metabolite” refers to a compound produced by cells. In some embodiments, a “metabolite” refers to a metabolic byproduct. In some embodiments, a “metabolite” refers to a waste product.

Exemplary metabolites include, but are not limited to, glutamine, glutamate, lactate, ammonium (NH₄⁺), sodium ions (Na⁺), potassium ions (K⁺), or calcium ions (Ca²⁺).

In some embodiments, the cell culture is contacted with the initial feed when the glutamine concentration is equal to or greater than 0.2 mmol/L, 0.25 mmol/L, 0.3 mmol/L, 0.35 mmol/L, 0.4 mmol/L, 0. 45 mmol/L, or 0.5 mmol/L. In some embodiments, the cell culture is contacted with the second medium when the glutamate concentration is equal to or greater than 3.35 mmol/L, 3.40 mmol/L, 3.45 mmol/L, 3.5 mmol/L, 3.55 mmol/L, or 3.6 mmol/L. In some embodiments, the cell culture is contacted with the second medium when the lactate concentration is equal to or greater than 2.7 g/L, 2.8 g/L, 2.9 g/L, 3.0 g/L, 3.1 g/L, 3.2 g/L, 3.3 g/L, or 3.4 g/L. In some embodiments, the cell culture is contacted with the second medium when the ammonium concentration is equal to or greater than 0.7 mmol/L, 0.8 mmol/L, 0.9 mmol/, 1.0 mmol/L, 1.1 mmol/L, 1.1 mmol/L, 1.2 mmol/L, 1.3 mmol/L, 1.35 mmol/L, or 1.4 mmol/L. In some embodiments, the cell culture is contacted with the second medium when the sodium ion concentration is equal to or greater than 96.5 mmol/L, 98 mmol/L, 100 mmol/L, 105 mmol/L, 110 mmol/L, 115 mmol/L, 120 mmol/L, 125 mmol/L, or 130 mmol/L. In some embodiments, the cell culture is contacted with the second medium when the potassium ion concentration is equal to or greater than 2.75 mmol/L, 2.85 mmol/L, 2.95 mmol/L, 3.05 mmol/L, 3.2 mmol/L, 3.35 mmol/L, or 3.5 mmol/L. In some embodiments, the cell culture is contacted with the second medium when the calcium ion concentration is equal to or greater than 0.05 mmol/L, 0.6 mmol/L 0.7 mmol/L, 0.8 mmol/L, 0.9 mmol/L, or 1.0 mmol/L.

In some embodiments, the cell culture is contacted with the second medium (e.g., a feed medium) when the concentration of a nutrient falls at or below a threshold value.

In some embodiments, the nutrient is a carbon source. In some embodiments, the carbon source is a sugar. In some embodiments, the sugar is glucose, galactose, fructose, mannose, lactose, sucrose, maltose, or trehalose. In some embodiments, the carbon source is pyruvate.

In some embodiments, the cell culture is contacted with the second medium when the glucose concentration is equal to or less than 0.1 g/L, 0.075 g/L, 0.05 g/L, 0.025 g/L, or 0.01 g/L.

In some embodiments, the nutrient is a nitrogen source.

In some embodiments, the nutrient is an amino acid. In some embodiments, the amino acid is alanine, arginine, asparagine, aspartic acid, cysteine, glutamine, glutamic acid, glycine, histidine, isoleucine, lysine, methionine, phenylalanine, proline, serine, threonine, tryptophan, tyrosine, or valine. In some embodiments, the amino acid is an essential amino acid. In some embodiments, essential amino acids include, but are not limited to, histidine, isoleucine, leucine, lysine, methionine, phenylalanine, threonine tryptophan, and valine.

In some embodiments, a nutrient is an antioxidant source. In some embodiments, the antioxidant nutrient is L-cysteine. In some embodiments, the antioxidant nutrient is N-acetyl-L-cysteine.

In some embodiments, the nutrient is a lipid. In some embodiments, the lipid is cholesterol. In some embodiments, the nutrient is a salt. In some embodiments, the salt is sodium, potassium, calcium, manganese, or magnesium. In some embodiments, the nutrient is a vitamin. In some embodiments, the vitamin is a B vitamin. In some embodiments, the vitamin is riboflavin, thiamine, or biotin. In some embodiments, the vitamin is vitamin A or vitamin E. In some embodiments, the nutrient is a mineral.

In some embodiments, act 215 comprises contacting the cell culture with a third medium.

In some embodiments, the third medium (e.g., a feed medium) is added when the concentration of a metabolite reaches or exceeds a threshold value. In some embodiments, the metabolite measured to determine the timing of act 215 is the same as the metabolite measured to determine the timing of act 210. In some embodiments, the metabolite measured to determine the timing of act 215 is a different metabolite than the metabolite measured to determine the timing of act 210.

In some embodiments, the third medium (e.g., a feed medium) is added when the concentration of a nutrient falls at or below a threshold value. In some embodiments, the nutrient measured to determine the timing of act 215 is the same as the nutrient measured to determine the timing of act 210. In some embodiments, the nutrient measured to determine the timing of act 215 is a different nutrient than the nutrient measured to determine the timing of act 210.

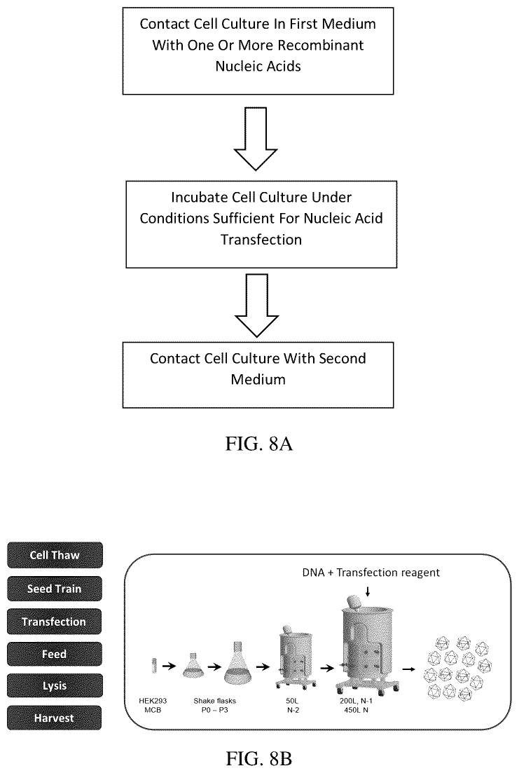

FIGS. 8A-B and 9 show non-limiting embodiments of process 200. In an embodiment, shown in FIG. 8A, a cell culture in a first medium is contacted with one or more recombinant nucleic acids. The cell culture is then incubated under conditions sufficient for nucleic acid transfection. The cell culture is then contacted with a second medium. In an embodiment, as shown in FIG. 8B, preparing the cell culture comprises thawing frozen cells and following a seed train protocol. The prepared cells are then transfected. The cells are then fed as described in this application. Once rAAV production is done, the cells are lysed and the rAAV is harvested.

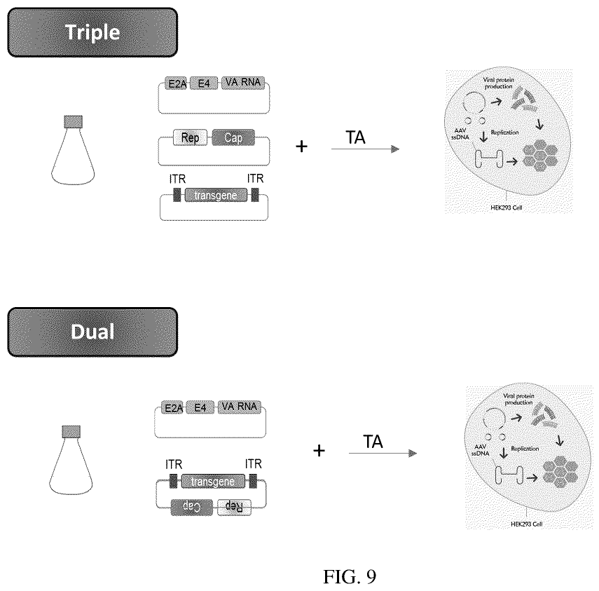

FIG. 9 shows non-limiting embodiments of transfection processes. In the top panel, three plasmids (Ad Helper, RepCap and Transgene flanked by ITRs) are transfected with the transfecting agent (TA) into HEK293 cells. In the lower panel, two plasmids (Ad Helper and a second plasmid comprising RepCap and Transgene flanked by ITRs) are transfected with the transfecting agent (TA) into HEK293 cells. In some embodiments, the respective genes are expressed and help in the replication and packaging of the AAV.

In some embodiments, act 220 comprises isolating rAAV from the cell culture. In some embodiments, isolating rAAV from the cell culture comprises harvesting the cell culture. In some embodiments, isolating rAAV from the cell culture comprises lysing the cells comprising the cell culture. In some embodiments, isolating rAAV from the cell culture comprises removing rAAV from the cell culture medium. In some embodiments, act 220 comprises preparing the isolated rAAV for delivery to a subject. In some embodiments, act 220 comprises purifying and/or sterilizing the isolated rAAV. In some embodiments, act 220 comprises preparing the rAAV for delivery to a human subject (e.g., to deliver a therapeutic gene to the subject to assist in the treatment of a disease or condition).

### AAV Production

Recombinant adeno-associated virus (rAAV) vectors are useful in gene therapy to deliver therapeutic genes to patient cells and tissue. An rAAV particle typically comprises a recombinant nucleic acid encapsidated within AAV capsid proteins to form an rAAV particle that can be administered to a subject. The recombinant nucleic (e.g., recombinant AAV genome) acid typically includes a heterologous gene of interest (e.g., encoding a therapeutic nucleic acid and/or protein) flanked by AAV inverted terminal repeat (ITR) sequences. In some embodiments, the AAV capsid proteins can be naturally occurring capsids of different AAV serotypes. For example, different AAV serotypes have different tissue tropisms and can be used to target different tissue types and associated diseases. In some embodiments, the AAV capsid proteins include one or more amino acid substitutions relative to naturally occurring capsid proteins.

Different manufacturing techniques can be used to produce rAAV particles. Typically, rAAV particles are assembled in host cells in culture (e.g., a cell culture in a bioreactor or other cell culture vessel). One or more nucleic acids encoding the recombinant AAV genome, AAV capsid proteins, and/or one or more Rep and helper genes are expressed in the host cell. The host cell is grown in culture (e.g., in a suspension culture, or on plates). The assembled rAAV is then isolated from the cell culture. The host cell can be a mammalian cell, an insect cell or other cell type. In some embodiments, a host cell is a producer cell.

### Transient Transfection

Transient transfection is one way of introducing heterologous genetic material to cells of interest. As used herein, “transfection” means nucleic acid transfection. Briefly, transfection comprises contacting a cell culture with one or more nucleic acids and a transfection reagent, also called a “transfecting agent.”

### Media

Aspects of the present disclosure provide methods of contacting a cell culture in a first medium with a second medium (e.g., a feed medium). In some embodiments, contacting a cell culture with a second medium is also referred to as “feeding” the cells. In some embodiments, medium is referred to as “feed.” In some embodiments, the first medium is the same as the second medium (e.g. feed medium). In some embodiments, the first medium is different from the second medium.

In some embodiments, the cell culture is contacted with a third medium (e.g., a feed medium) after 24 hours of incubation. In some embodiments, the first, second, and third media are the same medium. In some embodiments, the first and third media are the same medium. In some embodiments, the first and second medium are the same medium. In some embodiments, the second and third medium are the same medium. In some embodiments, the first medium, second medium (e.g., second medium, and third medium (e.g., third medium are each a different medium.

### Cell Culture

In some aspects, the present disclosure relates to methods of scaling up and/or increasing rAAV production by a cell culture.

In some embodiments, the cell culture produces adeno-associated virus (AAV) particles. In some embodiments, the cell culture produces recombinant AAV (rAAV) particles. In some embodiments, the presently described methods are combined with other methods to increase production of rAAV particles. In some embodiments, combining the presently-described methods with additional improvements in, for example feeding methods, leads to a 10-fold increase in AAV particle production or productivity. In some embodiments, successful scale-up of the entire manufacturing method is demonstrated from 250 mL reactors to 500 L bioreactors. In some embodiments, the presently disclosed method improves the production of rAAV particles relative to a current method. In some embodiments, the presently disclosed method increases production of rAAV particles relative to a current method. In some embodiments, the method of this application increases production of rAAV particles by 1.5 fold, 2 fold, 3 fold, 4 fold, 5 fold, 6 fold, 7 fold, 8 fold, 9 fold, or 10 fold relative to a current method.

In some embodiments, the method of this application improves the percent full capsids produced relative to a current method. In some embodiments, the method of this application increases production of full capsids by 1.5 fold, 2 fold, 3 fold, 4 fold, or 5 fold relative to a current method.

In some embodiments, the method of this application reduces the time required to produce levels of rAAV for harvest relative to a current method. In some embodiments, the method reduces the time by 6 hours, 12 hours, 18 hours, 24 hours, or 28 hours. In some embodiments, the method reduces the time by 1 day.

Recombinant AAVs

Naturally occurring AAV capsid proteins can be used to produce rAAVs for gene therapy. Different naturally occurring AAVs have different characteristics (including for example different tissue tropisms) and can be used for different indications. AAVs are highly prevalent within the human population (see Gao, G., et al., Clades of Adeno-associated viruses are widely disseminated in human tissues J Virol. 2004. 78 (12): p. 6381-8, and Boutin. S., et al., Prevalence of serum IgG and neutralizing factors against adeno-associated virus (AAV) types 1, 2, 5, 6, 8, and 9 in the healthy population, implications forgone therapy using AAV vectors. Hum Gene Ther. 2010. 21 (6): p. 704-12) and are useful as viral vectors. Many serotypes exist, each with different tropism for tissue types (see Zincarelli, C., et al., Analysis of AAV serotypes 1-9 mediated gene expression and tropism in mice after systemic injection. Mol Ther, 2008. 16 (6): p. 1073-80), which allows specific tissues to be preferentially targeted with appropriate pseudotyping. Some serotypes, such as serotypes 8, 9, and rh 10, transduce the mammalian body. See Zincarelli, C., et al. Analysis of AAV serotypes 1-9 mediated gene expression and tropism in mice after systemic injection. Mol Ther, 2008. 16 (6): p. 1073-80, Inagaki, K., et al., Robust systemic transduction with AAV9 vectors in mice: efficient global cardiac gene transfer superior to that of AAV8. Mol Ther, 2006. 14 (1): p. 45-53, Keeler, A. M., et al., Long-term correction of very long-chain acyl-coA dehydrogenase deficiency in mice using AAV9 gene therapy. Mol Ther, 2012. 20 (6): p. 1131-8, Gray, S. J., et al., Preclinical differences of intravascular AAV9 delivery to neurons and glia: a comparative study of adult mice and nonhuman primates. Mol Ther, 2011. 19 (6): p. 1058-69, Okada, H., et al., Robust Long-term Transduction of Common Marmoset Neuromuscular Tissue With rAAVI and rAAV9. Mol Ther Nucleic Acids, 2013. 2: p. e95, and Foust, K. D., et al., Intravascular AAV9 preferentially targets neonatal neurons and adult astrocytes. Nat Biotechnol, 2009. 27 (1): p. 59-65. AAV9 has been demonstrated to cross the blood-brain barrier (see Foust, K. D., et al., Intravascular AAV9 preferentially targets neonatal neurons and adult astrocytes. Nat Biotechnol, 2009. 27 (1): p. 59-65, and Rahim, A. A., et al., Intravenous administration of AAV2/9 to the fetal and neonatal mouse leads to differential targeting of CNS cell types and extensive transduction of the nervous system. FASEB J, 2011. 25 (10): p. 3505-18) that is inaccessible to many viral vectors and biologics. Certain AAVs have a payload of 4.7-5.0 kb (including viral inverted terminal repeats (ITRs), which are required in cis for viral packaging). See Wu, Z., H. Yang, and P. Colosi, Effect of genome size on AAV vector packaging. Mol Ther, 2010. 18 (1): p. 80-6 and Dong, J. Y., P. D. Fan, and R. A. Frizzell, Quantitative analysis of the packaging capacity of recombinant adeno-associated virus. Hum Gene Ther, 1996. 7 (17): p. 2101-12.

In some embodiments, rAAVs can include one or more variant AAV capsid proteins having one or more amino acid substitutions relative to a naturally occurring AAV capsid protein.

Accordingly, in some embodiments, the rAAV particles comprise AAV1, AAV2, AAV3, AAV4, AAV5, AAV6, AAV7, AAV8, AAV9, AAV10, AAV11, or AAV12 capsid proteins, or amino acid sequence variants thereof, or chimeras, or hybrids thereof.

In some embodiments, the rAAV particles comprise an rAAV genome. In some embodiments, an rAAV genome comprises a gene of interest flanked by inverted terminal repeats (ITRs). In some embodiments, a gene of interest comprises a therapeutic molecule, such as a therapeutic protein or therapeutic RNA. In some embodiments, a therapeutic molecule is an antibody, a protein, a peptide, an enzyme, or a ribozyme.

### Downstream Processing

In some embodiments, rAAV isolation comprises a further clarification and/or purification step. In some embodiments, rAAV isolation comprises a flocculation step. In some embodiments, a lysis agent, for example a detergent, can be used along with the flocculation agent.

In some embodiments, rAAV particles are further purified, for example using one or more affinity, ion exchange chromatography, and/or hydrophobic interaction chromatography steps, e.g., after clarification of an rAAV preparation.

In some embodiments, rAAV particles (e.g., after one or more purification steps) are added to a pharmaceutically acceptable solution.

The application also provides compositions comprising rAAV particles, and methods of administering the rAAV particles to a subject (e.g., a human subject having a condition that the therapeutic RNA and/or protein can help treat).

These and other aspects are illustrated by the following non-limiting examples.

### EXAMPLES

### Example 1. Scaling Up Using Industrial Mixer

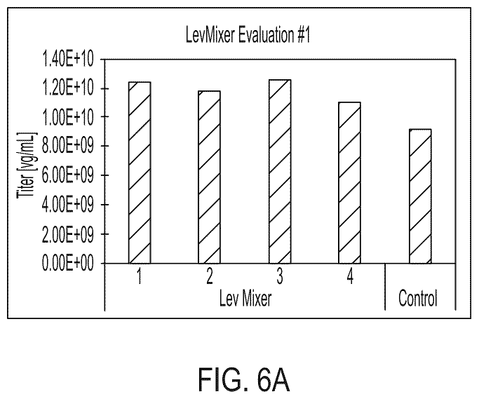

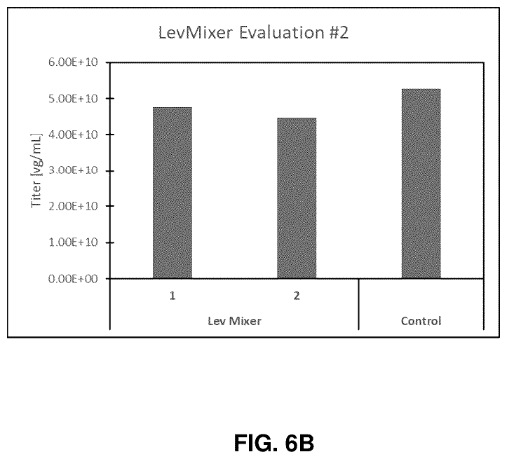

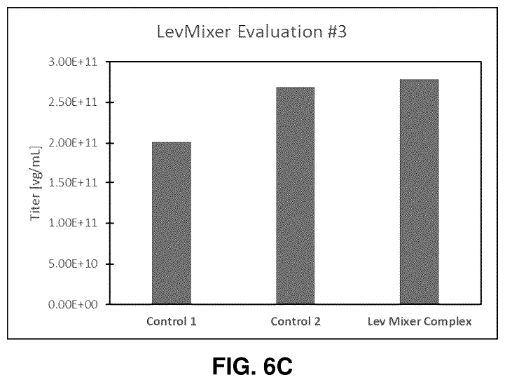

To facilitate use of larger transfection volumes without requiring multiple transfers of transfection complexes to a bioreactor housing a cell culture, an industrial mixer was used. A LevMixer® (Pall) was used to mix plasmids and transfection reagent in a first medium to generate transfection complexes. These transfection complexes were used to transfect HEK293 cells. Replicates using the transfection complexes formed in a LevMixer® were compared to a control using transfection complexes formed using a current method. Results are shown in FIGS. 6A-6C. Across multiple replicates, the transfection complexes formed in bags compatible with a LevMixer® resulted in improved AAV titers relative to transfection using prior method that does not involve transfection complex formation in the bags.

### Example 2. pH Monitoring of Cell Culture Indicates Surprising Opportunity to Enhance Viral Production

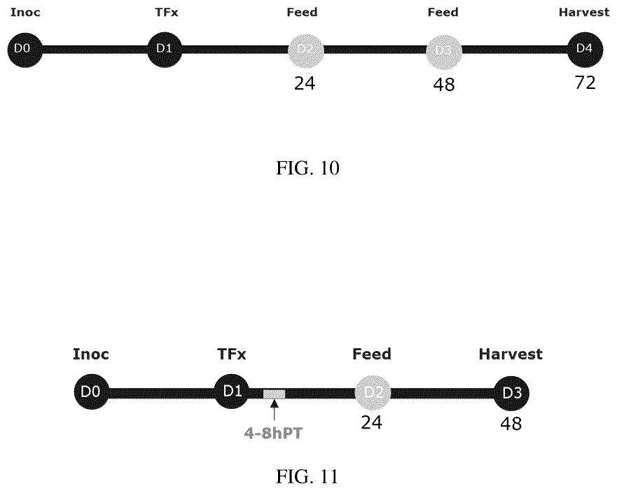

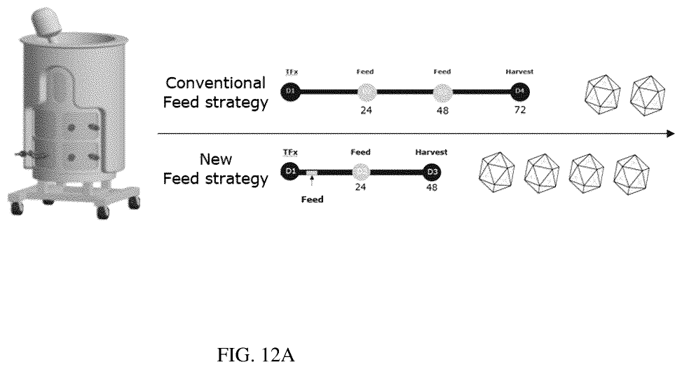

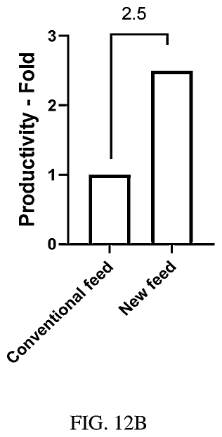

pH and CO2 levels were measured over time in a culture of HEK293 cells using an embodiment of the standard feeding procedure (see FIG. 10). PH levels were interpreted to indicate that the cells were either producing lactate or consuming lactate. Surprisingly, the measurements indicated that cells were consuming lactate within a few hours of transfection (TFx). Cells consume lactate when other carbon sources are limited in the growth medium. This suggested that providing additional medium during this time could improve production of viral particles. This experiment was subsequently repeated with a second medium provided at 4 hours post-transfection.

### Example 3 rAAV Production is Increased by Feeding at 4 Hours Post-Transfection

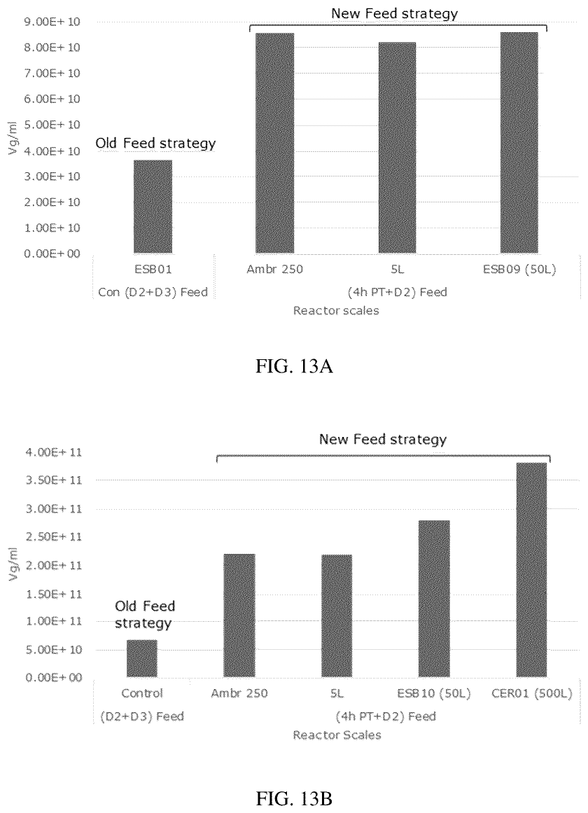

HEK293 cells were grown and transfected using the current method (see FIG. 11) and using a method further including providing media to the cells at 4 hours post-transfection. Three culture volumes were tested: 250 mL, 5L and 50 L. For the dual plasmid system, 500 L was also tested. Results are shown in FIG. 13A and B. Both a triple plasmid system (FIG. 13A) and a dual plasmid system (FIG. 13B) provided a second medium at 4 hours post-transfection show improvement over the current method.

### Example 4 Adding Second Medium 4 Hours Pre-Transfection is Counter-Productive

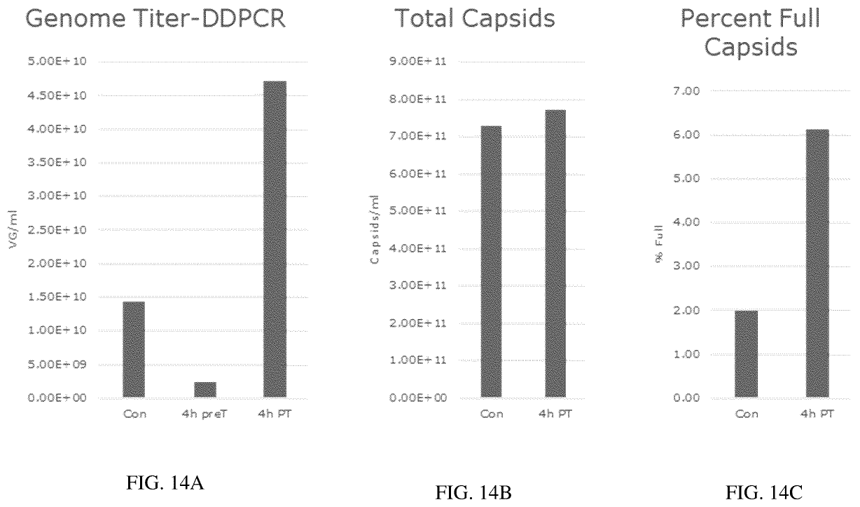

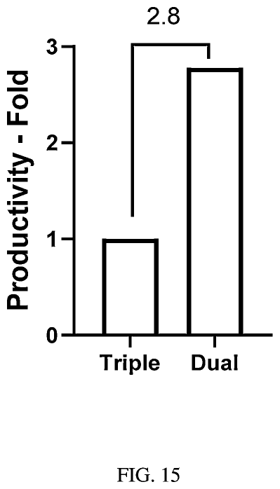

To evaluate whether providing additional media at another time would confer the same benefits as feeding at 4 hours post-transfection, 6×10^6 HEK293 cells were grown in 15 mL reactors. Cells were contacted with medium a) according to the current method (see FIG. 10), b) with additional medium provided at 4 hours pre-transfection, or c) 4 hours post-transfection. At 72 hours post-transfection, the cultures were harvested. Viral genomes were measured by digital droplet PCR (ddPCR). The number of capsids and percent full capsids were also measured using ELISA. Results are shown in FIGS. 14A-14C. As shown in FIG. 14A, providing additional medium 4 hours pre-transfection decreases viral particle production. As shown in FIGS. 14A-14C, providing additional medium at 4 hours post-transfection increases genome titer, total capsids, and percent full capsids, relative to the current method.

### Example 5. Providing Second Medium at 4 Hours Post-Transfection Allows for Harvest a Day Early

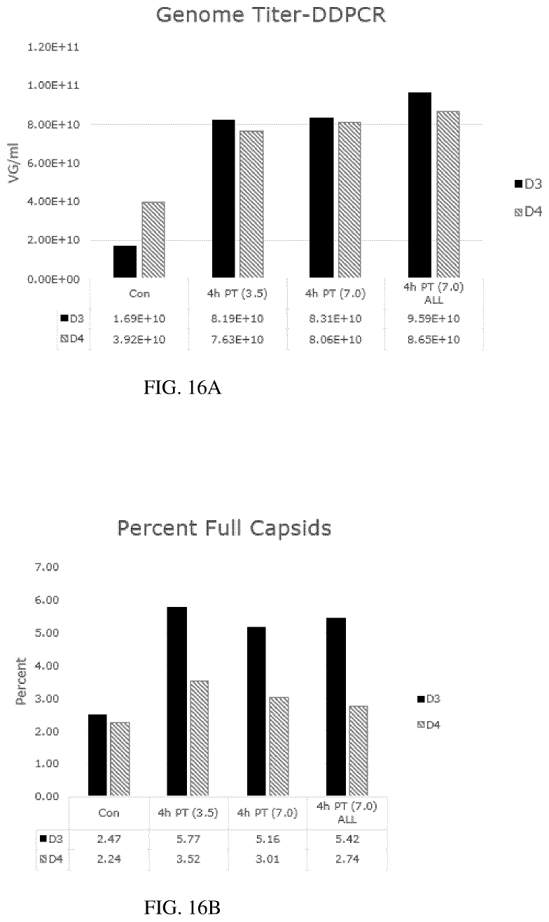

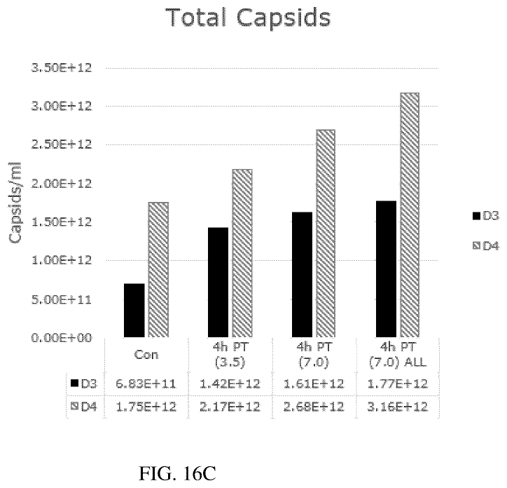

To further examine the effect of adding a second medium at 4 hours post-transfection, HEK293 cells were grown in 250 mL reactors and grown according to the current method (control or “con”), provided a single dose of second medium at 4 hours post-transfection (4 h PT (3.5)) and at 24 hours-and 48 hours-PT, provided a double dose of second medium 4 hours post-transfection (4 h PT (7.0)) and a single dose of second medium at 24 hours-and 48-hours PT, or provided a double dose of second medium at 4 hours-, 24-hours, and 48-hours PT. At 48 hours (D3) and 72 hours (D4) post-transfection, viral particles were harvested. Viral genome titer, percent full capsids, and total capsids were assessed as described in Example 3. Results are shown in FIGS. 16A-16C. Interestingly, providing a double dose of second medium did not improve genome titer, percent full capsid, or total capsids by much compared to single dose condition. Additionally, while harvesting at 72 hours post transfection increased the total number of capsids with the same genome titer, the percent full capsids was higher at 48 hours post-transfection compared to the control.

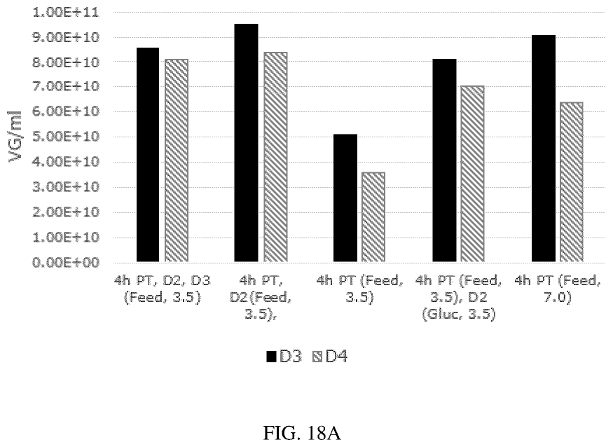

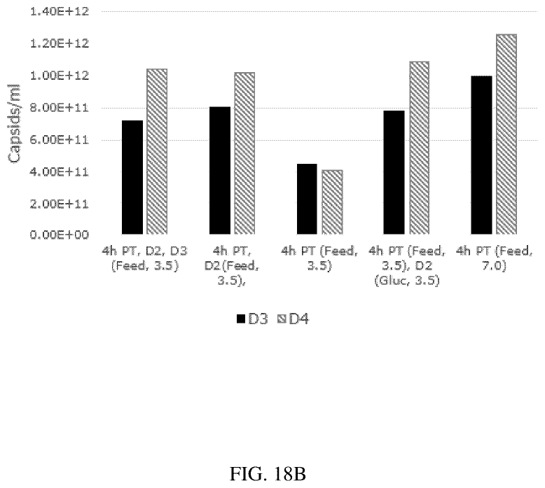

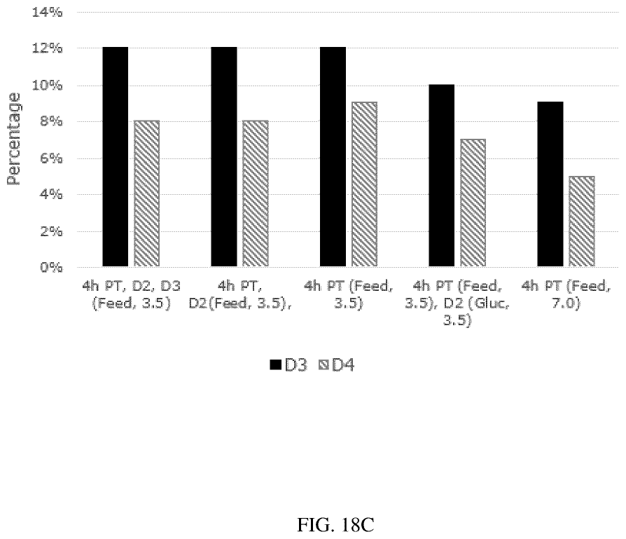

Similar experiments were done to assess the effect of omitting the feed step 24 hours post-transfection (D2) and 48 hours post-transfection (D3). Results are shown in FIGS. 18A-18C. Omitting the D3 feed resulted in comparable titers and percent full capsids compared to the control. Omitting both the D2 and D3 feed resulted in reduced titers compared to control. Adding glucose instead of media for the D2 feed was about as productive as adding medium at D2. Providing a double dose of medium at 4 hours post-transfection and omitting the D2 feed showed similar genome titers relative to a cell culture fed a single dose at both 4 hours post-transfection and at D2, but showed a decline in percentage of full capsids.

### Example 6. Providing Second Medium Between 4-8 Hours Provides the Most Benefit

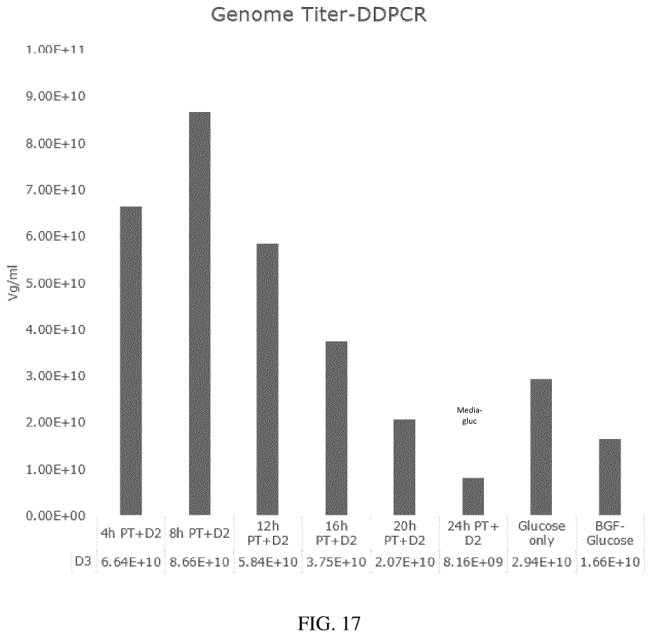

To determine the effect of feeding at other times post-transfection, HEK293 cells were grown in 250 mL reactors and contacted with a second medium at 4 hours post-transfection, 8 hours post-transfection, 12 hours post-transfection, 16 hours post-transfection, and 20 hours post-transfection. Cell culture volumes were between 180 and 220 mL. Cells were additionally provided a third medium at 24 hours post-transfection (D2). HEK293 cells in 250 mL reactors were also provided only glucose at 4 hours post-transfection, second medium without glucose at 4 hours post-transfection, or only a double dose of second medium at 24 hours post-transfection (D2). Viral particles were harvested at 48 hours post-transfection (D3). As shown in FIG. 17, providing second medium at 4 hours post-transfection or 8 hours post-transfection produced the highest viral genome titer. Providing second medium at 12 hours post-transfection was beneficial, but not as beneficial as providing second medium at 4-or 8-hours post-transfection.

### Example 7. Scalability of Combined Methods

The combined effects of the mixing and feeding methods described herein on rAAV production and scalability was tested. FIG. 7A shows a diagram depicting scaling up manufacturing from 250 mL reactors to 5 L reactors, 50 L reactors, and then to 500 L bioreactors. FIG. 7B shows the increase in productivity relative to a standard method (“conventional”) resulting from combining the presently-disclosed methods with a dual plasmid system (“+dual”), improved feeding method (“feed”), or both (“+dual+feed”). FIG. 7C shows the relative genome titer achieved by using the presently-described method in 250 mL reactors, 3L reactors, 5L reactors, 50 L reactors, or 500 L bioreactors. Values are normalized to the genome titer observed at 500 L. FIG. 7D shows the relative capsid titer achieved by using the presently-described method in 250 mL reactors, 3L reactors, 5L reactors, 50 L reactors, or 500 L bioreactors. Values are normalized to the capsid titer observed at 500 L.

The advantages associated with the methods of this disclosure was further demonstrated in multiple GMP runs (a production run manufactured according to cGMP guidelines), using product quantity and quality as attributes. As shown in Table 1, using a dual transfection system, the transfection complex mixing strategy and feed timing of about 4-8 hours post-transfection (e.g., 6 hours), high AAV titer was obtained along with an improved percentage of full capsids.

This demonstrated that usage of LexMixer® ensured a successful DNA complex formation and delivery to 500 L bioreactor. The data shows sustained the cell performance post transfection, maintenance of high productivity and product quality for the GMP runs from cell culture all the way to product recovery after downstream purification using cell lysis and chromatographic purification (e.g., anion exchange chromatography-based purification). DS corresponds to drug substance, comprising AAV vector comprising a transgene of interest. The percent full was assessed using vector an exemplary technique called mass photometry (MP).

TABLE 1

| | | Bioreactor | | DS |
|---|---|---|---|---|
| | Bioreactor AAV | total viral | DS % | recovery |
| GMP-500 L | titer (vg/mL) | genome | full (MP) | (%) |
| GMP1 & GMP2 | >4E11 | >2E17 | >70% | >40% |

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method comprising:
a) contacting a cell culture in a first medium with one or more recombinant nucleic acids;
b) incubating the cell culture for 4-8 hours under conditions sufficient for nucleic acid transfection; and
c) contacting the cell culture of b) with a second medium.

2. The method of claim 1, wherein one of the one or more recombinant nucleic acids encodes a recombinant AAV (rAAV) genome.

3. The method of claim 2, wherein the rAAV genome comprises a gene of interest flanked by inverted terminal repeats (ITRs).

4-15. (canceled)

16. The method of claim 1, further comprising contacting the cell culture with a third medium 24 hours after the start of (b).

17-21. (canceled)

22. The method of claim 1, wherein the cell culture produces adeno-associated virus (AAV) particles.

23-33. (canceled)

34. A method, comprising
a) incubating one or more recombinant nucleic acids and one or more transfection reagents in a vessel in a first medium under conditions sufficient for complex formation; and
b) contacting a cell culture with a volume of the first medium using pressure, wherein the volume is at least 25 L, and wherein the pressure is greater than one atmosphere (atm).

35-40. (canceled)

41. The method of claim 34, wherein steps (a) and (b) combined last about 10 minutes.

42-45. (canceled)

46. The method of claim 34, wherein one of the one or more recombinant nucleic acids encodes a recombinant AAV (rAAV) genome.

47. The method of claim 46, wherein the rAAV genome comprises a gene of interest flanked by inverted terminal repeats (ITRs).

48-75. (canceled)

76. The method of claim 34, wherein the method further comprises
a) contacting a cell culture in a first medium with one or more recombinant nucleic acids;
b) incubating the cell culture for 4-8 hours under conditions sufficient for nucleic acid transfection; and/or
c) contacting the cell culture of b) with a second medium.

77. The method of claim 1, further comprising isolating rAAV from the cell culture after step (b) or step (c).

78. A method comprising:
a) incubating one or more recombinant nucleic acids and one or more transfection reagents in a vessel in a first medium under conditions sufficient for complex formation; and
b) contacting a cell culture with a volume of the first medium, wherein the volume is at least 25 L;
c) incubating the cell culture for 4-8 hours under conditions sufficient for nucleic acid transfection; and
d) contacting the cell culture of c) with a second medium.

79. The method of claim 78, wherein (a) produces a plurality of transfection complexes, and wherein the plurality of complexes is transferred to a bioreactor comprising the cell culture as a single volume.
